(19) United States
(12) Patent Application Publication
(10) Pub. No.: US 2012/0088298 A1
(43) Pub. Date: Apr. 12, 2012

(54) IKKALPHA AND IKKBETA SPECIFIC INHIBITORS

(75) Inventors: Jun LI, Danbury, CT (US); Xiang LI, New Milford, CT (US); Katrina Mary CATRON, Middlebury, CT (US)

(73) Assignee: BOEHRINGER INGELHEIM PHARMACEUTICALS, INC., Ridgefield, CT (US)

(21) Appl. No.: 13/329,607

(22) Filed: Dec. 19, 2011

Related U.S. Application Data
(63) Continuation of application No. 12/849,359, filed on Aug. 3, 2010, now abandoned.
Application No. 12/849,359 is a division of application No. 11/241,490, filed on Sep. 30, 2005, now abandoned.
(60) Provisional application No. 60/614,652, filed on Sep. 30, 2004.

(51) Int. Cl. C12N 15/63; C07H 21/02

(52) U.S. Cl. 435/320.1; 536/24.5

(57) ABSTRACT

A method for modulating NF-κB dependent gene transcription in a cell comprised of modulating IKKα and IKKβ protein and protein activity in the cell. The present invention also provides siRNA compositions and methods thereof for modulating NF-κB dependent gene transcription.

### RELATED APPLICATIONS

This application is a Divisional of U.S. application Ser. No. 11/241,490, filed Sep. 30, 2005, which claims priority to U.S. Provisional Application No. 60/614,652, filed Sep. 30, 2004, the contents of which are incorporated herein in their entireties.

### BACKGROUND OF THE INVENTION

This invention relates to the field of, inflammatory diseases and autoimmune diseases and the treatment thereof through the modulation of IKKα and IKKβ activity.

### BACKGROUND INFORMATION

### Roles of IKKα and IKKβ in Inflammation

IKKα and IKKβ are kinases that phosphorylate IκB. The phosphorylation of IκB is understood in the art to be a major triggering event in regulation of the NF-κB pathway. The NF-κB or nuclear factor κB is a transcription factor that plays a critical role in inflammatory diseases by inducing the expression of a large number of proinflammatory and anti-apoptotic genes. These include cytokines such as IL-1, IL-2, IL-11, TNF-α and IL-6, chemokines including IL-8, GRO1 and RANTES, as well as other proinflammatory molecules including COX-2 and cell adhesion molecules such as ICAM-1, VCAM-1, and E-selectin. Pahl H L, (1999) Oncogene 18, 6853-6866; Jobin et al, (2000) Am. J. Physiol. Cell. Physiol. 278: 451-462. Under resting conditions, NF-κB is present in the cytosol of cells as a complex with IκB. The IκB family of proteins serve as inhibitors of NF-κB, interfering with the function of its nuclear localization signal (see for example U. Siebenlist et al, (1994) Ann. Rev. Cell Bio., 10: 405). Upon disruption of the IκB-NF-κB complex following cell activation, NF-κB translocates to the nucleus and activates gene transcription. Disruption of the IκB-NF-κB complex and subsequent activation of NF-κB is initiated by degradation of IκB. Activators of NF-κB mediate the site-specific phosphorylation of two amino terminal serines in each IκB which makes nearby lysines targets for ubiquitination, thereby resulting in IκB proteasomal destruction. NF-κB is then free to translocate to the nucleus and bind DNA leading to the activation of a host of inflammatory response target genes. Baldwin, A., Jr., (1996) Annu Rev Immunol 14: 649-683, Ghosh, S. et al, (1998) Annu Rev Immunol 16, 225-260. Recent evidence has shown that NF-κB subunits dynamically shuttle between the cytoplasm and the nucleus but a dominant acting nuclear export signal in IκBα ensures their transport back to the cytoplasm.

The phosphorylation of IκB is a major triggering event in regulation of the NF-κB pathway. Since the abnormal regulation of the NF-κB pathway is known to correlate with inflammatory disease, the regulation of IκB phosphorylation is understood as an important area for disease intervention. The search for the kinase responsible for the inducible phosphorylation of IκB has been one of the major focuses in the NF-κB field. IκB phosphorylation is mediated by a high molecular weight signalsome complex consisting of at least three components: two IκB kinases IKKα, IKKβ and a non-catalytic regulatory subunit NEMO (reviewed in Mercurio, F. et al, (1999) Oncogene, 18: 6163-6171; Barkett, M. et al, (1999) Oncogene, 18: 6910-6924; Karin, M., (1999) Oncogene, 18: 867-6874). Studies on IKKα- or IKKβ-deficient mouse embryonic fibroblast cells (MEF) show that IKKβ is essential for signal induced IκBα phosphorylation while IKKα was found to be dispensable for this initial phase of canonical NF-κB activation (Li, Zw et al, J Exp Med. 1999 Jun. 7; 189(11):1839-1845; Hu, Y. et al, (1999) Science, 284, 316-320.). However, IKKα null MEFs still failed to express NF-κB target genes in response to pro-inflammatory stimuli (Li, X. et al (2002) J. Biol. Chem., 277, 45129-45140), which uncovered a nuclear role for IKKα in the canonical NF-κB activation pathway (Anest, V. et al (2003) Nature, 423, 659-663). The regulatory role of IKKα and IKKβ in the NF-κB pathway are discussed in U.S. patent application Ser. No. 10/446,045 the contents of which are incorporated herein.

### Gene Silencing

Experimental procedures can be used to specifically inactivate or silence a target gene or inhibit the activity of its gene product. Inhibition of protein activity can be brought about at the level of gene transcription, protein translation or post translational modifications. For instance, the activity of a protein can be inhibited by directly inhibiting the activity of the protein such as altering a catalytic domain or alternatively by reducing the amount of the protein in the cell by reducing the amount of mRNA encoding the protein. In each case the level of protein activity in the cell is reduced. Various techniques can be used to knock down the activity of a protein and these include knockout technologies (antibodies, antisense RNA, and RNA interference) and compounds that specifically inhibit the protein activity. Antisense RNAs directed to IKKα has been reported for use in the inhibition of IKKα expression. U.S. Pat. No. 6,395,545.

RNA interference (RNAi) is a technique that can be used to knockdown the activity of genes and their protein products in a specific manner. RNAi was first used in the Nematode worm Caenorhabditis elegans as a response to double stranded RNA (dsRNA) that resulted in the gene knockdown specific manner. Fire, A. et al, (1998) Nature, 391: 806-811. RNAi is a process whereby a double stranded RNA (dsRNA) of a sequence that is homologous to a target gene can be used to cause the degradation of messenger RNA (mRNA) transcribed from that target gene. Sharp, P. A., (2001) Genes Dev., 15: 485-490. Initiation of gene silencing or gene inactivation occurs upon recognition of dsRNA by the cells machinery that convert the silencing trigger to 21-25 nucleotides RNAs. Hannon, (2002) Nature, 418: 244-250.

The mediators of sequence-specific messenger RNA degradation are typically 21- and 22-nucleotide small interfering RNAs (siRNAs) generated by ribonuclease III cleavage from longer dsRNAs. In vitro synthesized 21-nucleotide siRNA duplexes specifically suppress expression of endogenous and heterologous genes in different mammalian cell lines, including human embryonic kidney and HeLa cells. Elbashir S. et al, (2001) Nature, 411: 494-498. Therefore, 21-nucleotide siRNA duplexes provide a new tool for studying gene function in mammalian cells and may be used as gene-specific therapeutics. However, effective gene silencing is only caused by a subset of siRNAs complementary to the mRNA target. McManus M T et al, (2002) J. Immunol. 169: 5754-60. Thus, design of multiple siRNA oligos and extensive testing are required to obtain a potent siRNA oligo. McManus M T et al, (2002) J. Immunol. 169: 5754-60.

The ability to specifically knock down expression of a target gene by siRNA has many benefits. For example siRNA could be used to mimic true genetic knockout animals to study gene function. There have been reports of using siRNA for various purposes including the inhibition of luciferase gene expression in human cells, (see US Patent Application No. 2002/0132788); HIV-1 Cellular receptor CD4 (Sharp et al, (2002) Nature Medicine, 8: 681-686); HIV accessory genes, vif and nef (Nature Advance Online Publication, Jun. 26, 2002 (doi:10.1038/nature00896); HPV E6 and E7 gene expression. Jiang M., Oncogene, (2002), 21:6041-6048); Subtype- and species-specific knockdown of protein kinase C (Inci N. et al, Biochem. Biophys. Res. Commun., (2002) 298: 738-743.

### BRIEF SUMMARY OF THE INVENTION

The present invention provides siRNA oligonucleotides that specifically inhibit IKKα and IKKβ and that can be used to inhibit the expression of NF-κB dependent genes. The sequences encoding the IKKα and IKKβ specific siRNA's can also be incorporated into retroviral vectors. The NF-κB dependent gene can be selected from IL-6, IL-8, IL-2, Cox-2, ICAM-1, VCAM-1, GM-CSF, tumor necrosis factor, Gro-1, Rantes, and serum amyloid A.

Another aspect of the present invention relates to a method for inhibiting the expression of NF-κB dependent genes, said method comprised of the steps of administering to a cell an IKKα specific inhibitor and an IKKβ specific inhibitor. In the preferred method of the invention the IKKα specific inhibitor and the IKKβ specific inhibitor are administered simultaneously. Alternatively, the IKKα specific inhibitor can be administered first and then the IKKβ specific inhibitor or vice versa.

Another aspect of the invention relates to the discovery that a synergistic inhibition of NF-κB dependent gene expression can be obtained by administration to a cell of a combination of IKKα and IKKβ specific inhibitors and that the level of inhibition of NF-κB dependent gene expression obtained is greater than observed when IKKα and IKKβ inhibitors are administered to a cell independently.

Another aspect of the invention relates to a method for treating autoimmune or inflammatory diseases comprised of the steps of administering to a patient in need thereof of a therapeutic amount an IKKα specific inhibitor and an IKKβ specific inhibitor.

Another aspect of the invention provides an siRNA oligonucleotide comprised of SEQ ID. No: 1 directed to IKKα and SEQ. ID. No. 3 directed to IKKβ.

Another aspect of the invention provides for an expression vectors such as retroviral vectors incorporating SEQ. ID No. 1 and/or SEQ. ID. No. 3.

Another aspect of the invention is a retroviral vector of SEQ. ID No. 1 or SEQ. ID. No. 3 wherein comprised of a 5′ LTR, a selective gene, a Polymerase-III, RNA gene promoter, and a 3′LTR.

Another embodiment of the invention provides for a method for inhibiting the expression of NF-κB dependent cells, said method comprised of the steps of administering to a cell an IKKα specific inhibitor and an IKKβ specific inhibitor wherein the specific inhibitors can be administered simultaneously. Alternatively, the IKKα specific inhibitor is administered first and then the IKKβ specific inhibitor or vice versa.

Another embodiment provides a method for treating autoimmune and inflammatory disease to a patient in need thereof comprised of the steps of administration of an IKKα and an IKKβ specific inhibitor.

Another embodiment of the invention provides a method for modulating NF-κB dependent gene transcription by administration of an IKKα specific inhibitor and an IKKβ specific inhibitor wherein the specific inhibitors are comprised of siRNA directed to IKKα and IKKβ cDNA sequence.

### BRIEF DESCRIPTION OF THE FIGURES

FIG. 1 shows the retrovirus expressed hairpin IKKα siRNA oligo sequence.

FIG. 2 shows IKKα mRNA expression in HeLa cells infected with IKKα siRNA retrovirus.

FIG. 3 shows a western blot showing that retrovirus-expressed IKKα siRNA inhibits IKKα protein but not IKKβ protein.

FIGS. 4A and 4B show IL-6 and IL-8 mRNA expression in HeLa cells expressing IKKα siRNA or GL2 (control siRNA) by retroviral vectors.

FIG. 5 shows the retrovirus expressed hairpin IKKβ siRNA oligo sequence.

FIG. 6 shows IKKβ mRNA expression in HeLa cells infected with IKKβ siRNA retrovirus.

FIG. 7 shows a western blot showing that retrovirus-expressed IKKβ siRNA inhibits IKKβ protein but not IKKα protein.

FIGS. 8A and 8B show IL-6 and IL-8 mRNA expression in HeLa cells expressing retrovirus expressed GL2 (control siRNA) or IKKβ siRNA.

FIGS. 9A, 9B, 9C and 9D show_IKKα, IKKβ, IL-6 and IL-8 mRNA expression in HeLa cells expressing GL-2 or IKKα siRNA, with or without additional IKKβ siRNA oligo transfection.

FIGS. 10A, 10B, 10C and 10D show IKKα, IKKβ, IL-6, and IL-8 mRNA expression in HeLa cells stably transfected with retroviruses expressing GL-2 or IKKα or IKKβ siRNA, with or without additional IKKα or IKKβ siRNA oligo transfection.

### BRIEF DESCRIPTION OF THE SEQUENCES

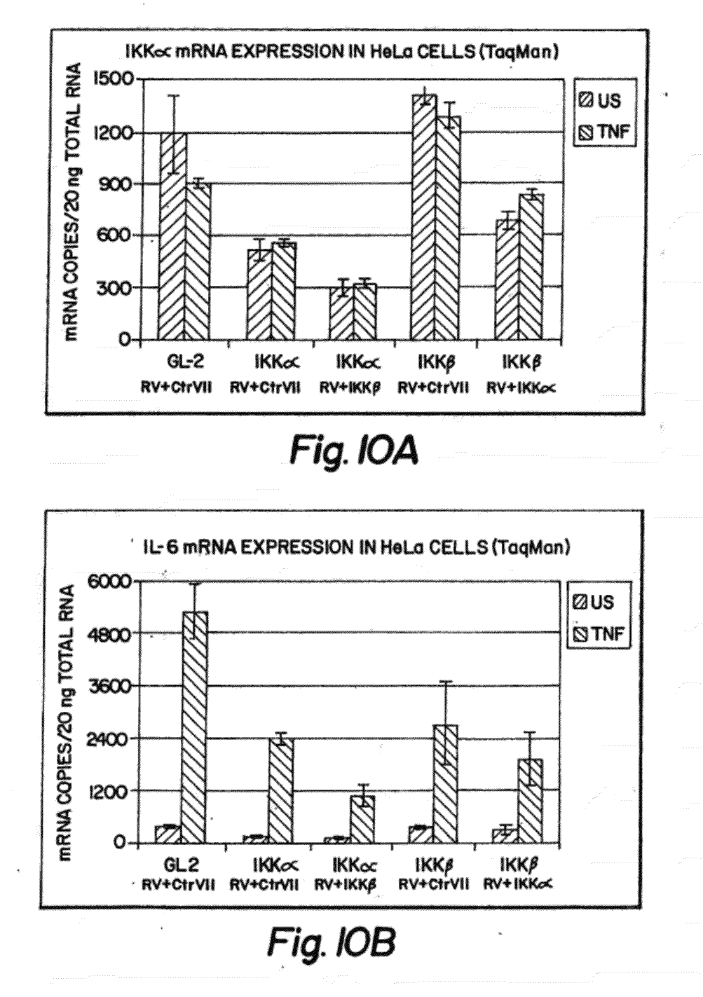

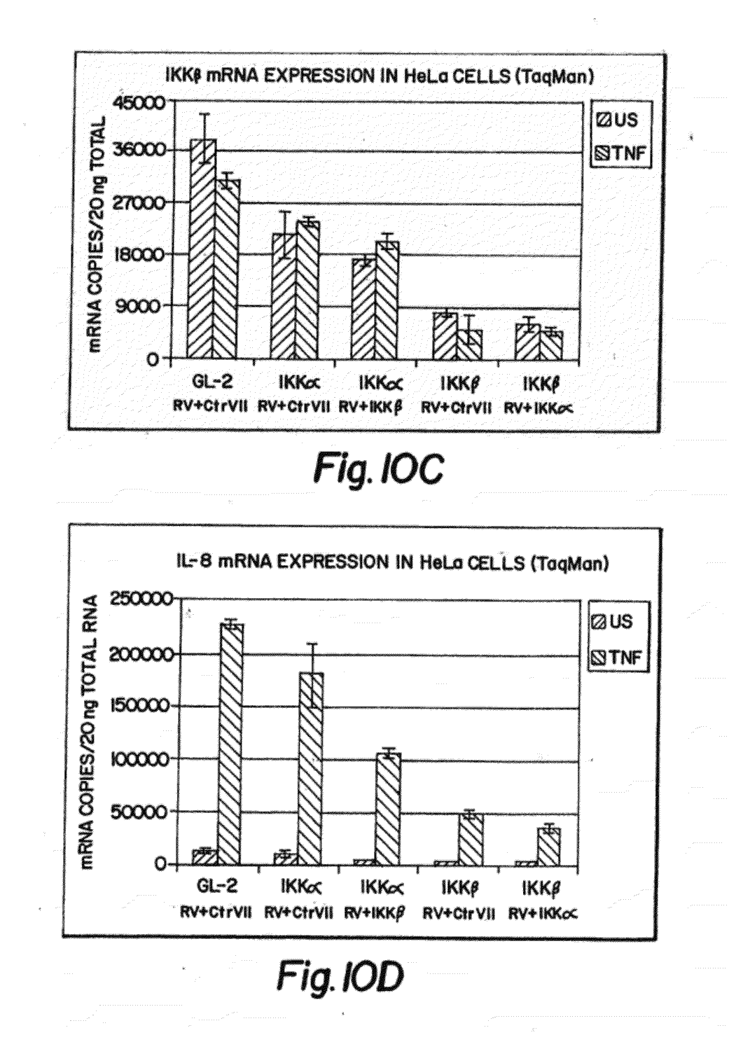

SEQ ID. No. 1 is the sequence for the siRNA hairpin of IKKα (Forward oligo).
SEQ ID. No. 2 is the sequence for the siRNA hairpin of IKKα (Reverse oligo).
SEQ ID. No 3 is the sequence for the siRNA hairpin of IKKβ (Forward oligo).
SEQ ID. No 4 is the sequence for the siRNA hairpin of IKKβ (Reverse oligo).
SEQ ID No. 5 is the siRNA oligo targeting the luciferase gene known as GL2-sense.
SEQ ID No. 6 is the siRNA oligo targeting the luciferase gene known as GL2-antisense.
SEQ. ID. No. 7 is the TaqMan forward primer of IKKα.
SEQ. ID. No. 8: is the TaqMan reverse primer of IKKα.
SEQ ID. No. 9: is the TaqMan forward primer of IKKβ.
SEQ ID. No. 10 is the TaqMan reverse primer of IKKβ.
SEQ ID. No. 11 is the TaqMan probe sequence for IKKα labeled with a reporter.
SEQ ID. No. 12 is the TaqMan probe sequence for IKKβ labeled with a reporter
SEQ ID. No. 13 is a control siRNA sequence from the IKKα siRNA inverted sequence used in FIG. 9.
SEQ ID No. 14 is a control siRNA sequence (Ctr. VII) used for transient transfection in FIG. 10.
SEQ ID No. 15 is the IKKα siRNA sequence used for transient transfection in FIG. 10.
SEQ ID No. 16 is the IKKβ siRNA sequence used for transient transfection in FIG. 10.
SEQ ID No. 17 is the human IKKα cDNA sequence.
SEQ ID. No. 18 is the human IKKβ cDNA sequence.

### DETAILED DESCRIPTION OF THE INVENTION

### I. General Description

The present invention provides a method for modulating NF-κB dependent gene transcription, said method comprised of the step of modulating IKKα and IKKβ protein activity in a cell. The level of IKKα and IKKβ activity in a cell can be modulated upward or downward. The level of IKKα and IKKβ activity is preferentially modulated downward. One embodiment of the invention is based in part on the demonstration that the use of a dual IKKα and IKKβ specific inhibitor in TNFα stimulated human cells results in the modulation of genes under the influence of NF-κB.

The present invention employs a dual siRNA for use in modulating the level of IKKα and IKKβ protein activity in the cell. SiRNA oligonucleotides directed to IKKα and IKKβ specifically hybridize nucleic acids encoding IKKα and IKKβ interfere with IKKα and IKKβ gene expression. Accordingly, IKKα and IKKβ proteins levels are reduced and the total level of IKKα and IKKβ activity in the cell is reduced. Since IKKα and IKKβ have been shown to play a role in triggering the NF-κB pathway (Table I; Li, X. et al, (2002) J. Biol. Chem., 277: 45129-45140), which functions in the inflammatory response, compounds that have the property of being able to specifically and effectively inhibit IKKα are understood to be helpful in the treatment of autoimmune and inflammatory diseases.

### II. Definitions

Unless defined otherwise, the scientific and technological terms and nomenclature used herein have the same meaning as commonly understood by a person of ordinary skill in the art to which this invention pertains.

Nucleotide sequences are presented herein by a single strand, in the 5′ to 3′ direction, from left to right, using the one letter nucleotide symbols as commonly used in the art and according with the recommendations of the IUPAC-IUB Biochemical Nomenclature Commission (1972).

The term “IKKα” as it is used herein refers to the alpha subunit of the IκB kinase complex. IKKα is a kinase that phosphorylates IκB, NF-κB p100 or other protein substrates.

The term “IKKβ” as is it used herein refers to the beta subunit of the IκB kinase complex. IKKβ is a kinase that phosphorylates IκB, NF-κB p100 or other protein substrates.

The term “gene transcription” as it is used herein means a process whereby one strand of a DNA molecule is used as a template for synthesis of a complementary RNA by RNA polymerase.

The term “DNA” as used herein refers to polynucleotide molecules, segments or sequences and is used herein to refer to a chain of nucleotides, each containing the sugar deoxyribose and one of the four adenine (A), guanine (G) thymine (T) or cytosine (C).

The term “RNA” as used herein refers to polynucleotide molecules, segments or sequences and is used herein to refer to a chain of nucleotides each containing the sugar ribose and one of the four adenine (A), guanine (G) uracil (U) or cytosine (C).

The term “oligo” as used herein means a short sequence of DNA or DNA derivatives typically 8 to 35 nucleotides in length. An oligonucleotide can be derived synthetically, by cloning or by amplification. The term “derivative” is intended to include any of the above described variants when comprising an additional chemical moiety not normally a part of these molecules. These chemical moieties can have varying purposes including improving solubility, absorption, biological half life, decreasing toxicity and eliminating or decreasing undesirable side effects.

The term “RNAi” as used herein generally refers to the RNA interference process for a sequence-specific post-transcriptional gene silencing or gene knockdown by providing a double-stranded RNA (dsRNA) that is homologous in sequence to the targeted gene. Small interfering RNAs (siRNAs) can be synthesized in vitro or generated by ribonuclease III cleavage from longer dsRNA and are the mediators of sequence-specific mRNA degradation. The term “siRNA duplex” as used herein is meant to refer to a duplex of an oligonucleotide complexed with its reverse complement (antisense) sequence. The 5′ end dTdT overhang is included in both the sense and the reverse complement strands. The SEQ ID No's provided herein refer to the sense strand used in the complex. The antisense strand portion of these duplexes has not been included as separate Sequence listings.

The term “Expression vector” as defined herein can include adenovirus vectors, Lentivurs vectors, and non-virus based vectors containing RNA polymeraselll promoter.

The term “retrovirus” as used herein means a class of viruses that have their genetic material in the form of RNA and use reverse transcriptase to translate their RNA into DNA. Retroviruses are known in the art and are engineered to express siRNA's of interest. Retroviruses will typically contain of a 5′ LTR, a selective gene such as puromycin or GFP, a Polymerase-III, H1 or U6 RNA gene promoter, and a 3′LTR.

The term “modulating IKKα activity” or “modulating IKKβ activity” as used herein means either inhibiting (decreasing) or stimulating (increasing) the level of activity of IKKα or IKKβ protein in a cell (collectively IKK). IKKα activity can be modulated by to modification of the levels and/or activity of the IKKα protein, or by modification of the level of IKKα gene transcription and/or IKKα structure such that the level of IKKα protein activity in the cell is modulated. Likewise, IKKβ activity can be modulated by modification of the levels and/or activity of the IKKβ protein, or by modification of the level of IKKβ gene transcription and/or IKKβ activity structure such that the level of IKKβ protein activity in the cell is modulated. In the context of the present invention, inhibition is the preferred form of modulation.

The term “autoimmune and inflammatory disease” as used herein means diseases that are associated with autoimmune and inflammatory conditions such as osteoarthritis, reperfusion injury, asthma, multiple sclerosis, Guillain-Barre syndrome, Crohn's disease, ulcerative colitis, psoriasis, graft versus host disease, systemic lupus erythematosus, rheumatoid arthritis, Alzheimer's disease, toxic shock syndrome, insulin-dependent diabetes, acute and chronic pain as well as symptoms of inflammation and cardiovascular disease, stroke, myocardial infarction alone or following thrombolytic therapy, thermal injury, adult respiratory distress syndrome (ARDS), multiple organ injury secondary to trauma, acute glomerulonephritis, dermatoses with acute inflammatory components, acute purulent meningitis or other central nervous system disorders, Grave's disease, myasthenia gravis, scleroderma and atopic dermatitis.

The term “protein” as used herein means isolated naturally occurring polypeptides, or recombinantly produced proteins. Means for preparing such proteins are well understood in the art. Proteins may be in the form of a secreted protein, including truncated or mature forms. Proteins may optionally be modified to include an additional amino acid sequence which contains secretory or leader sequences, pro-sequences, sequences which aid in purification, such as multiple histidine residues, or an additional sequence for stability during recombinant production. The proteins of the present invention are preferably provided in an isolated form, and preferably are substantially purified. A recombinantly produced version of a protein, including the secreted protein, can be substantially purified using techniques described herein or otherwise known in the art, such as, for example, by the one-step method described in Smith et al, Gene, 67:31-40 (1988). Proteins of the invention also can be purified from natural, synthetic or recombinant sources using techniques described herein or otherwise known in the art.

The term “gene knockdown” as used herein refers to the reduction in the activity of a gene. The terms “gene silencing” or “gene inactivation” are considered to have the same meaning as the terms used herein.

The term “proinflammatory gene” as used herein refers to any gene that is induced upon an inflammatory response through the NF-κB pathway. Examples of proinflammatory genes include but are not limited to beta inhibin, IL-8, IL-6, interferon stimulated protein, TNF-induced protein, Cox2, GRO1 oncogene, CD44, interleukin 11, and superoxide dismutase.

The term “specific inhibitor” as used herein means an inhibitor that inhibits one protein more than another protein. For example, a potential inhibitor of IKKα is considered to be specific for IKKα over another IKKβ protein when there is preferably at least 10 to 100 fold or greater and most preferably about 1000 fold difference in inhibition of IKKα compared to IKKβ.

The term “NF-κB dependent gene transcription” as used herein means genes that are either upregulated or downregulated in response to the level of NF-κB activity in a cell. Such genes include, but are not limited to IL-6, IL-8, IL-2, intercellular adhesion molecule 1, interferon stimulated protein, Cox2, IL-11, GRO1 and superoxide dismutase. NF-κB dependent genes are also discussed in US 2002/0156000: Barnes et al. (1997) New England J. Med. 336: 1066-1071; Pahl H L, Oncogene, (1999), 18, 6853-6866 incorporated herein by reference.

The term “delivery” as used herein refers to the introduction of a foreign molecule (i.e. nucleic acid small molecule inhibitor) in cells.

The term “treating” as used herein means the prevention, reduction, partial or complete alleviation or cure of a disease.

Using the present invention it is possible to observe the function of IKKα. In addition, specific siRNA oligos directed to IKKα that have been designed and tested in human cells show a reduction in the expression of proinflammatory genes and NF-κB target genes with their use. These siRNA and equivalent compounds may have therapeutic value in the treatment of autoimmune and inflammatory disease as described herein. It is therefore understood that compounds that inhibit either IKKα expression or IKKα protein activity also have therapeutic value.

The term “administration” as used herein means the introduction of a foreign molecule (i.e. nucleic acid, small molecule inhibitor) into a cell. The term is intended to be synonymous with the term “delivery”.

### III. Specific Embodiments

a. The IKKα Specific siRNA

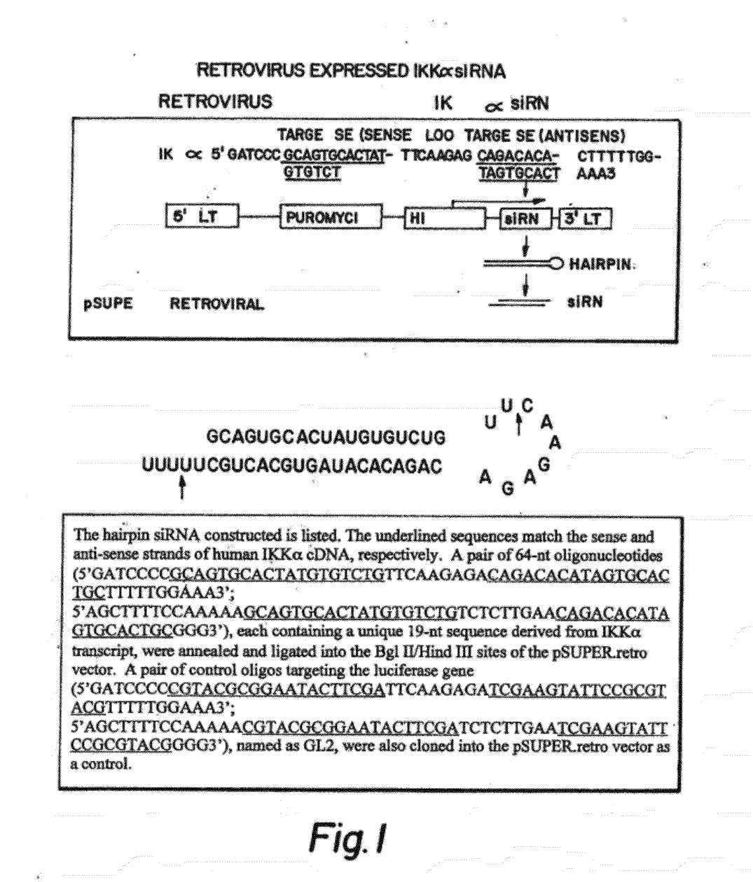

The present invention provides a hairpin loop siRNA that specifically inhibits IKKα. The hairpin loop of the sense strand is made up of a 64 nucleotide sequence as shown in SEQ ID. No. 1. The siRNA sequence may also be incorporated into a retroviral insert for use in a retroviral expression system. FIG. 1 shows the configuration of the siRNA of SEQ ID. No. 1 in retroviral system.

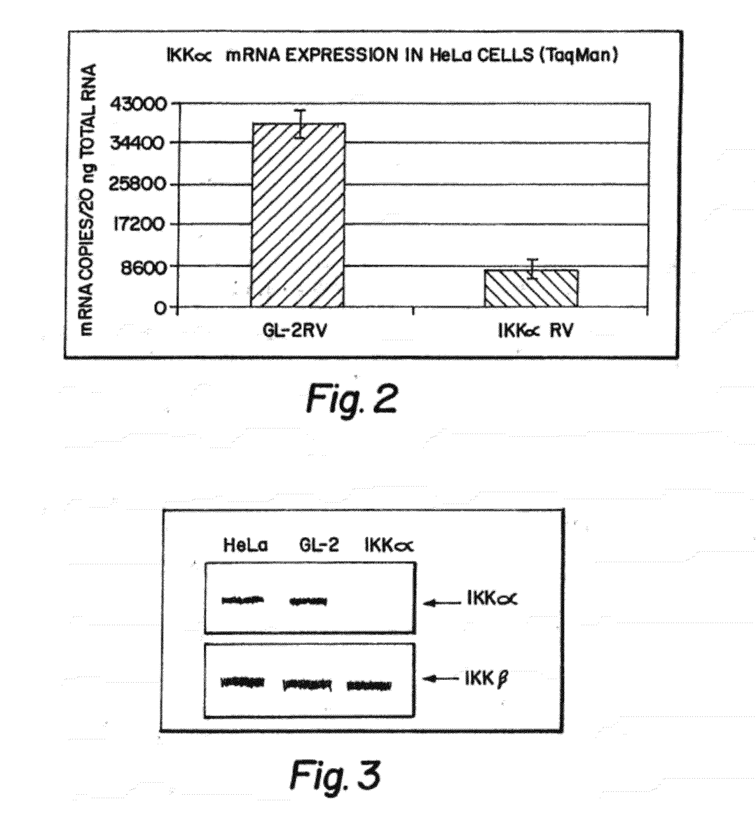

FIG. 2 shows the activity of the IKKα retrovirus in HeLa cells. HeLa cells were infected with pSUPER retroviruses containing IKKα and GL-2 siRNA hairpin sequences. The total RNAs were isolated from HeLa cells and TaqMan RT-PCR was performed to detect IKKα mRNA expression. FIG. 2 shows that HeLa cells that are transfected with IKKα retrovirus exhibit a significantly reduced level of IKKα expression.

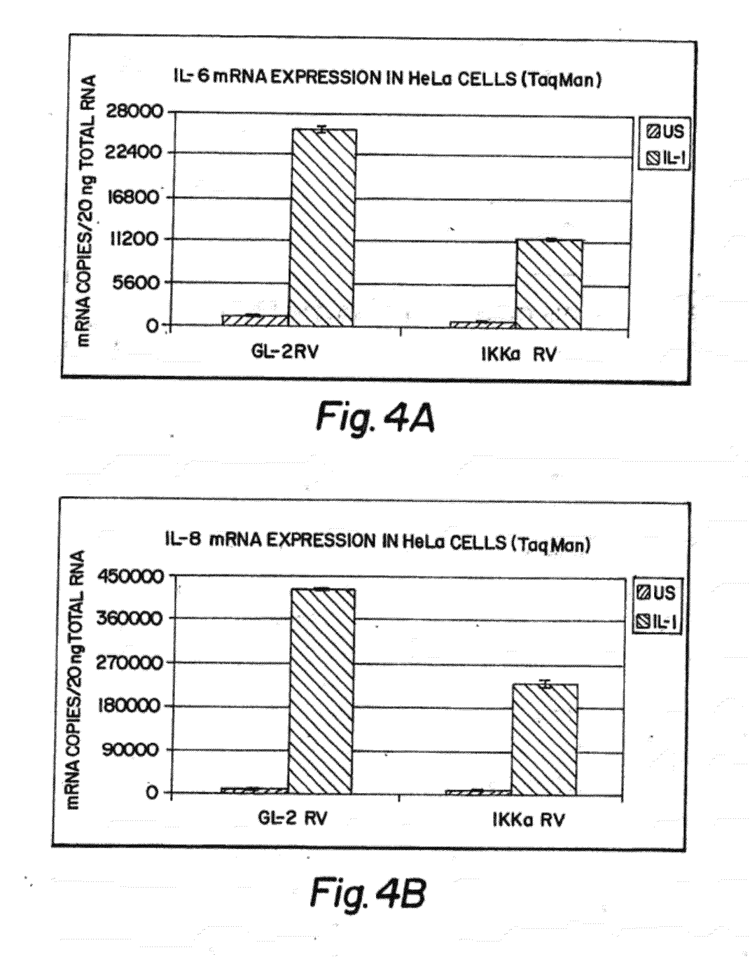

FIG. 3 is a Western Blot analysis showing that IKKα siRNA inhibits IKKα but not IKKβ protein. HeLa cells were stably infected with retrovirus expressing GL2 or IKKα siRNA. Cells were lysed and run through Western-blot analysis using anti-IKKα or anti-IKKβ to antibodies.

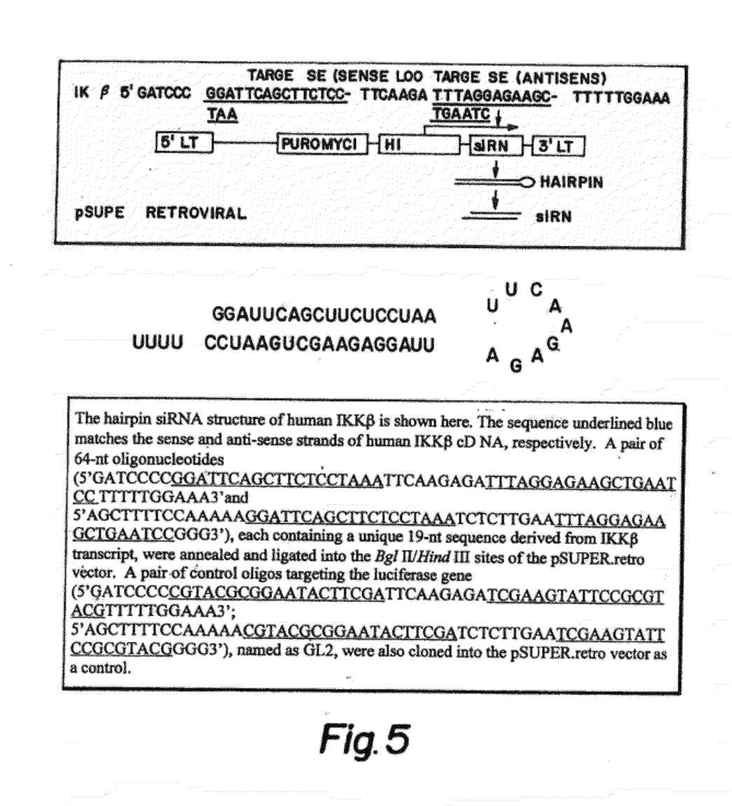

FIGS. 4A and 4B show the reduction in IL-6 and IL-8 expression induced in IL-1 stimulated cells stably infected with retrovirus expressing GL2 or IKKα siRNA.

b. The IKKβ Specific siRNA

The present invention also provides a hairpin loop siRNA that specifically inhibits IKKβ. The hairpin loop is made up of a 64 nucleotide sequence as shown in SEQ ID. No. 3. The siRNA sequence may also be incorporated into a retroviral insert for use in a retroviral expression system.

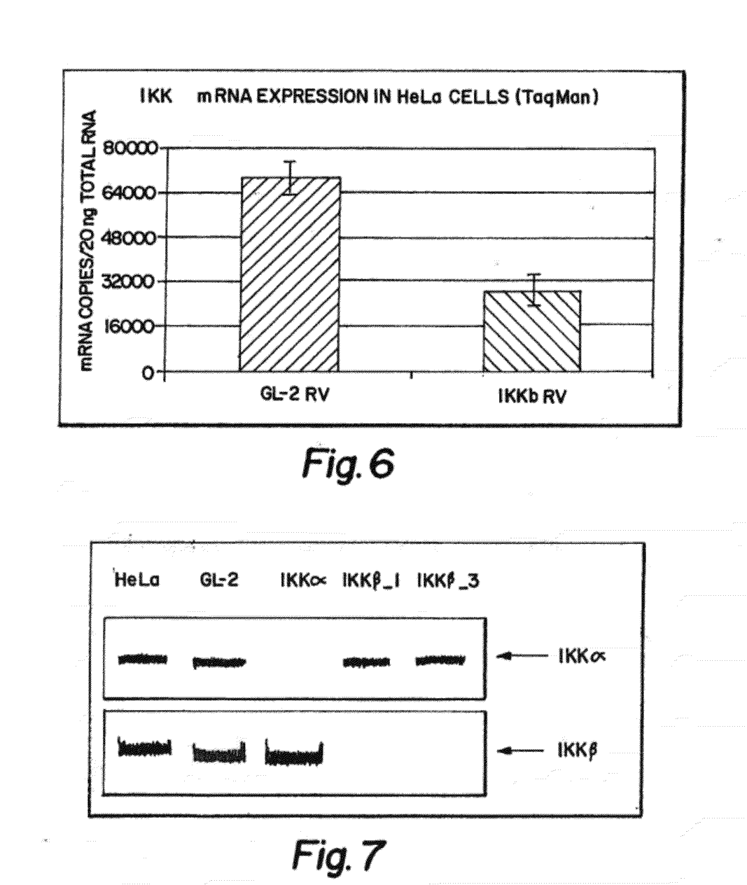

FIG. 5 shows the configuration of the siRNA of SEQ ID. No. 3 in a retroviral system.

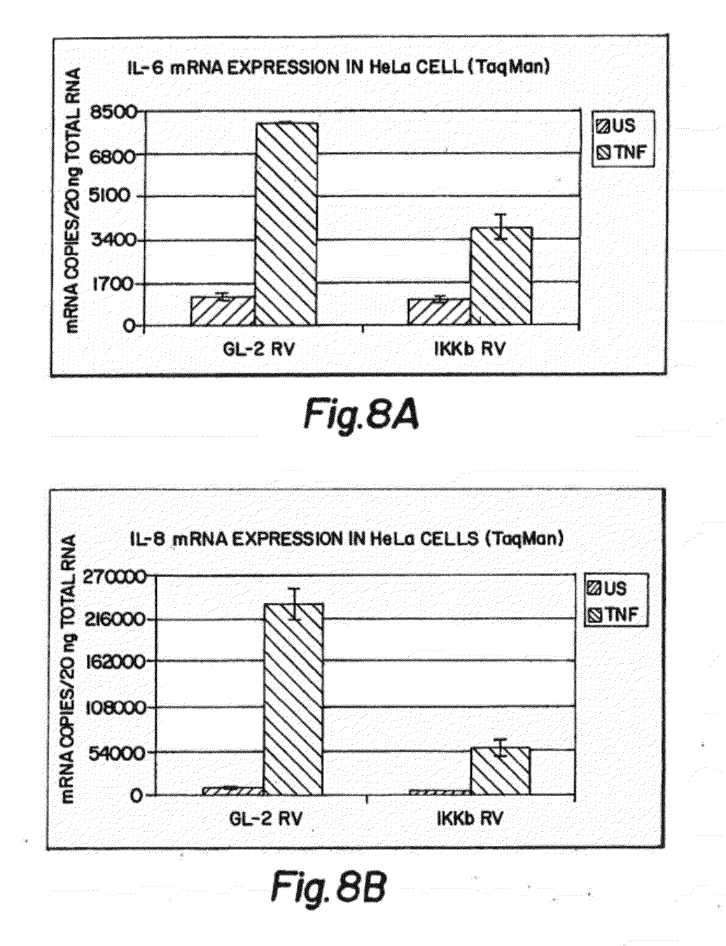

FIG. 6 shows the activity of the IKKβ specific inhibitor. HeLa cells were infected with pSUPER retroviruses containing IKKβ or GL-2 siRNA hairpin sequence. The total RNAs were isolated from infected HeLa cells and TaqMan RT-PCR was performed to detect IKKβ mRNA expression. FIG. 6 shows that the HeLa cells infected with IKKβ have significantly reduced expression of IKKβ.

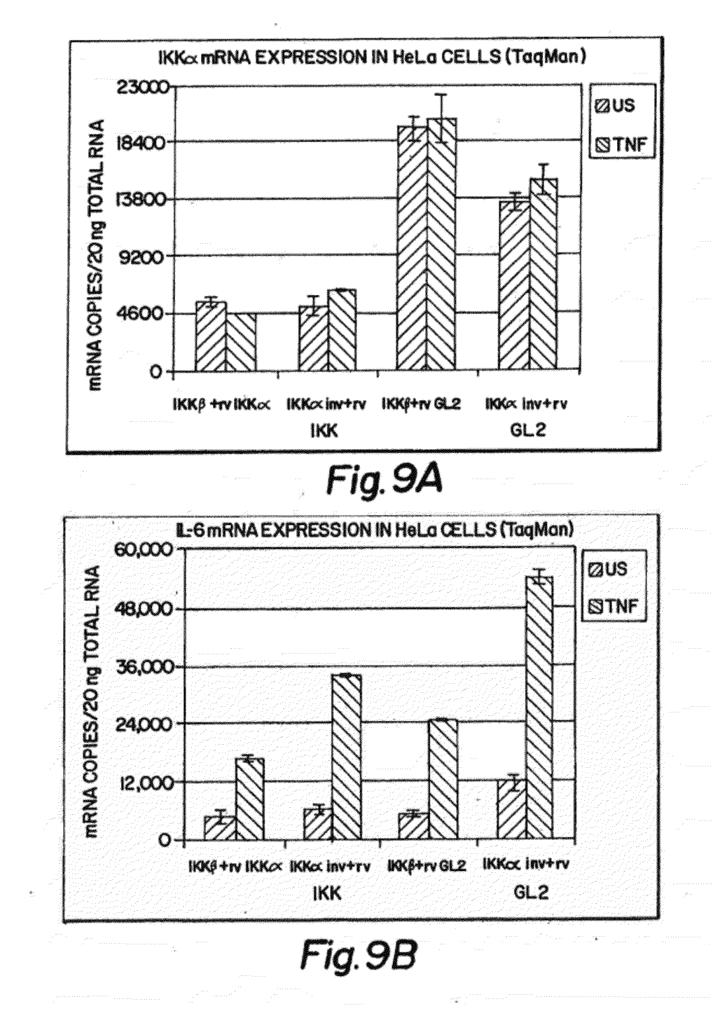

FIG. 7 shows Western Blot analysis of IKKβ siRNA inhibition of IKKβ protein but not IKKα protein. HeLa cells were stably infected with retrovirus expressing GL2 or IKKα siRNA. Cells were lysed and run through Western-blot analysis using anti-IKKα or anti-IKKβ antibodies.

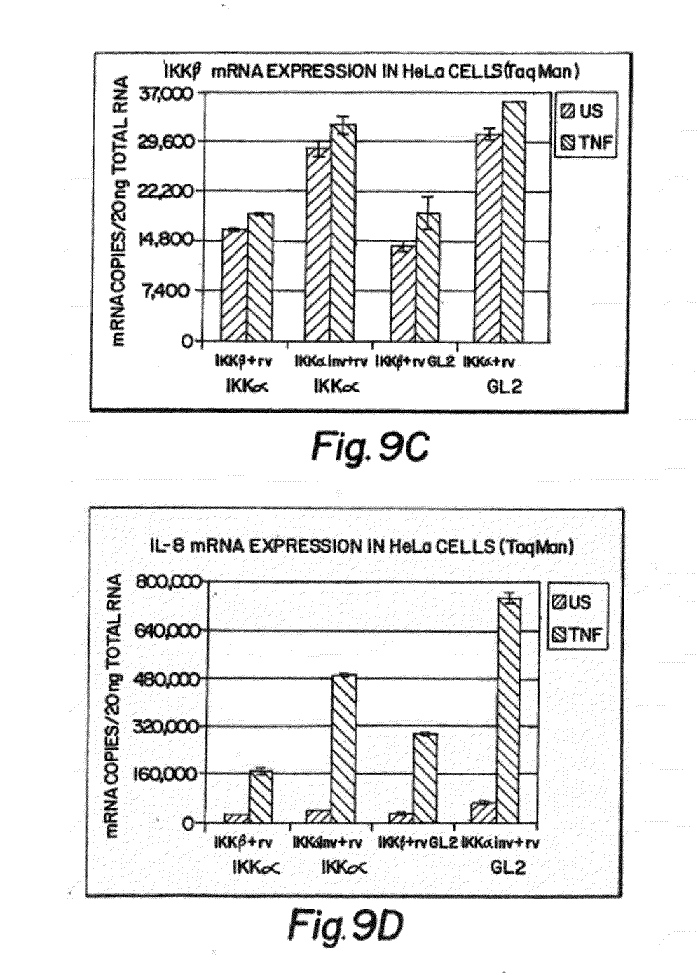

FIGS. 8A and 8B show_the reduction in IL-6 and IL-8 induced in IL-1 stimulated cells stably infected with retrovirus expressing GL2 and IKKβ siRNA.

c. Method for Inhibiting NF-κB Dependent Genes Using IKKα and IKKβ Specific Inhibitors

The present invention also provides a method for inhibiting the expression of NF-κB dependent genes by administration to a cell of a specific inhibitor of IKKα and a specific inhibitor of IKKβ. A stronger inhibitory effect on NF-κB dependent gene expression can be obtained than though use by specifically blocking either IKKα or IKKβ alone.

IKKα and IKKβ specific inhibitors can be siRNA directed to IKKα and IKKβ. Alternatively, the IKKα and IKKβ inhibitors can be small molecule inhibitors that are specific to IKKα and IKKβ respectively. IKKβ specific inhibitors can be found in U.S. patent application Ser. No. 10/453,175, the contents of which are incorporated herein.

We have shown that retrovirus-expressed hairpin IKKα siRNA stably and specifically suppressed IKKα mRNA (FIG. 2) and protein expression (FIG. 3). IKKα hairpin siRNA also inhibits TNF-induced IL-6 and IL-8 expression (FIGS. 4A and 4B). Thus, hairpin IKKα siRNA is a specific IKKα inhibitor for treating inflammatory diseases. We have also shown that retrovirus-expressed hairpin IKKβ siRNA stably and specifically suppressed IKKβ mRNA (FIG. 6) and protein expression (FIG. 7). IKKβ hairpin siRNA also inhibits TNF-induced IL-6 and IL-8 expression (FIGS. 8A and 8B). Thus hairpin IKKβ siRNA is a specific IKKβ inhibitor for treating inflammatory diseases.

Using the retrovirus-infected cells in which IKKα protein expression has been stably suppressed, it is shown that dual inhibition of IKKα and IKKβ by siRNA shows more effective inhibition of IL-6 and IL-8 expression than single inhibition of IKKα and IKKβ. As shown in FIGS. 9A, 9B, 9C and 9D HeLa cells were stably transfected with retroviruses expressing GL2 (control) or IKKα siRNA. The stably transfected cells were further transiently transfected with IKKα inv (control) sequence SEQ ID No. 13 annealed to its reverse compliment or IKKβ siRNA, comprised of the oligo of SEQ ID No. 16 and its reverse compliment to obtain single and dual inhibition of IKKα and IKKβ expression. The mRNA expression of IKKα, IKKβ, IL-6 and IL-8 were quantified by Taqman real-time RT-PCR. This result is further confirmed by using dual inhibition of IKKα and IKKβ in cells in which IKKβ is stably suppressed by IKKβ siRNA viruses (FIGS. 10A, 10B, 10C and 10D). In FIG. 10 HeLa cells were stably transfected with retroviruses expressing GL2 (control) or IKKα or IKKβ hairpin siRNA. The stably transfected cells were further transiently transfected with control CtrlVII siRNA made of an oligo of SEQ ID No. 14 and its reverse compliment, plus the 2-nucleotide 3′ overhang composed of (2′-deoxy) thymidine, or IKKα siRNA (SEQ ID No. 15 and its reverse compliment), or IKKβ siRNA, (SEQ ID No. 16 and its reverse compliment) to obtain single and dual inhibition of IKKα and IKKβ expression. The 2-nucleotide 3′ overhang composed of (2′-deoxy)thymidine exists in all siRNA duplexes. The mRNA expression of IKKα, IKKβ, IL-6 and IL-8 were quantified by Taqman real-time RT-PCR.

Thus, we provide a novel method of inhibiting the NF-κB pathway by dual inhibition of IKKα and IKKβ.

Another way to practice the invention is generating a stable IKKβ-silenced human cell line or human tissues for studying the function of IKKβ. Alternatively, a stable IKKα silenced human cell line or human tissues can be used for studying the function of IKKα.

Preferred aspects of embodiments of the present invention are described in the following examples, which are not to be construed as limiting.

The method of the invention can comprise modulating NF-κB dependent gene expression in a cell by administration of siRNA directed to IKKα and IKKβ. RNA interference is a method whereby siRNA can be used to knockdown or reduce the level of expression of a specific gene. SiRNA specifically directed to IKKα and IKKβ can be administered to cells in order to knockdown IKKα and IKKβ protein activity in the cell and to reduce the expression of NF-κB proinflammatory genes. SiRNA can be designed according to the technique described by Tuschl, described as follows. Elbashir, S M et al, Nature, 2001, 411, 494-498. SiRNA that can efficiently knockdown a gene can be obtained by using siRNA duplexes composed of 21 nt sense and 21 nt antisense strands paired in a manner to have a 2-nt 3′ overhang. The sequence of the 2-nt overhang is thought to make a contribution to the specificity of the target recognition restricted to the unpaired nucleotide adjacent to the first base pair. 2-Deoxynucleotides are used in the 3′ overhang.

The targeted region is selected from the human cDNA beginning at about 100 nt downstream of the start codon. The target sequence for IKKα is SEQ ID No. 20 and the target sequence for IKKβ is SEQ ID No. 18. Sequences can be searched for AA(N19)TT with approximately 40-60% G/C content. AA(N19) should match exactly the sequence of sense cDNA. The sequence of the sense siRNA corresponds to (N19)TT or N21, respectively. N19 exactly matches the sequence of sense cDNA. A blast search should be performed on the selected siRNA against genebank full-length genes and ESTs to ensure that only one gene is targeted. The sequence of the siRNA should be selective to the target sequence.

### General Methods

A. Preparation of the siRNA Duplexes

The siRNA duplexes used for delivery to cells can be prepared as follows. Approximately 0.02 to 0.2 μM of the synthetic siRNAs can be used for delivery to various types of cells such as HeLa cells, Jurkat T cells, lymphocytes, HUVEC cells and fibroblasts. SiRNAs can be obtained from a number of sources including Dharmacon (Lafayette, Colo.) and Ambion (Austin, Tex.). The siRNA can be prepared by synthesizing the sense and antisense strand 21-nt oligos, followed by annealing of the single stranded oligos. The siRNA can be incubated, pelleted and quantified using UV spectroscopy methods understood and used in the art.

B. Delivery of siRNA to cells and transfection of siRNA duplexes

Delivery of siRNA to cells can be performed according to cell transfection methods commonly used in the art. Elbashir S M et al, Nature, 2001, 411, 494-498; McManus M T et al, J. Immunol. 2002, 169:5754-60; Barton G M et al, Proc. Natl. Acad. Sci. (2002) 99:14943-5. Delivery of siRNA can be performed on various types of tissue culture cells. Preferably tissue culture cells of autoimmune or inflammatory significance such as lymphocytes, epithelium cells and endothelial cells should be used. More specifically cells such as HeLa cells, Jurkat T cells, lymphocytes, HUVEC cells and fibroblasts can be used. SiRNA can be delivered to tissue and organisms as well. Lewis D L et al, Nat. Genet. (2002) 32:107-8; McCaffrey A P et al, Nature (2002) 418:38-39.

Various transfection reagents can be used for siRNA delivery such as lipid-mediated transfection, electroporation or viral infection. In the preferred method the transfection reagent is OLIGOFECTAMINE™ available from Invitrogen (Carlsbad, Calif.). Transfection efficiencies should be between 40 and 100%.

For each sample between about 1 to 10 μg of siRNA duplex and about 100 μl of Opti-MEM are mixed. In a separate tube 1 volume of Oligofectamine and 4 volumes of Opti-MEM are incubated for 10 to 15 minutes at room temperature. The samples are then mixed and incubated for another 20 to 25 minutes at room temperature. Then 16 volumes of fresh Opti-MEM are added. SiRNA-transfection reagent is added to cultured cells (40 to 50% confluent). The cells are seeded for about 24 hours prior to transfection in antibiotic-free medium using culture techniques commonly used in the art.

A knockdown effect should be found between 1 to 5 days after delivery of the siRNA. The amount of knockdown is generally 40 to 100% of normal mRNA levels, and most preferably 60 to 100% of normal mRNA levels.

C. Treatment of Cells with a Proinflammatory Agent

In order to measure the extent of inhibition of NF-κB dependent proinflammatory genes, proinflammatory agents are administered to the cell. Acceptable proinflammatory agents are those that induce expression of proinflammatory genes in the NF-κB pathway. Proinflammatory agents include but are not limited to TNFα, IL-1 and LPS. The preferred proinflammatory agent is TNFα. It is understood that other proinflammatory agents may effect expression of NF-κB dependent genes. The stimulation time and the amount of proinflammatory agent that is used will vary according to the agent used but will be an amount sufficient to elicit a measurable proinflammatory response. TNFα is added to the cells at 1 to 10 ng/ml for 30 minutes to 24 hours. Typically, the proinflammatory agent is added before the measurement of proinflammatory genes is taken.

### D. Preparation of RNA and PCR Primers

The level of gene knockdown or inhibition of gene transcription can be measured by analysis of mRNA from total RNA samples. Total RNA can be prepared between about 24 and 72 hrs after delivery of siRNA using methods known to those skilled in the art. [www.invitrogen.com/transfection]. Preferably total cellular RNA is isolated from tissue or cell samples using the RNeasy™ kit and Rnase-Free DNase Set Protocol from Qiagen (Valencia, Calif.) according to the manufacturer's instructions.

### E. TaqMan Real-Time PCR Procedures

PCR analysis can be used to analyze the isolated RNA and quantify the effects of the IKKα and IKKβ inhibitor on the transcription of NF-κB dependent genes. PCR primers and/or probes used for the measurement of the transcription level of these genes can be prepared using techniques that are commonly used in the art. PCR primers should be designed for the amplification of the cDNA sequence from genes of interest. Software can be used to assist in designing primers specific for target genes. Preferred software is Primer Express 1.5 Software (Applied Biosystems (Foster City, Calif.). Probes can be labeled with reporter agents such as the fluorescent dye, FAM (6-carboxyfluorescein) at the 5′ end and a fluorescent dye quencher TAMRA (6-carboxy-tetramethyl-rhodamine) at the 3′ end. Other reporter agents commonly used in the art such as P³², S³⁵ fluorescein and Biotin can also be used. The specificity of PCR primers can be tested under normal PCR conditions in a thermal cycler prior to PCR quantitation. Total cellular RNA isolated from tissue or cell samples is used in reverse transcription (RT) reactions.

A “standard curve” can be constructed by plotting the Cₜ vs. the known copy numbers of the template in the standard. According to the standard curve, the copy numbers for all unknown samples are obtained automatically. To determine the copy numbers of the to target transcript, a human genomic DNA (Clontech, Palo Alto, Calif.) can be used to generate a standard curve. The copy numbers of genomic DNA template are calculated according to the molecular weight of human diploid genome [3×10⁹ bp=3×10⁹×660 (M.W.)=2×10¹² g], and then 1 μg/μl genomic DNA is converted into 2.4×10⁶ copy numbers based upon the Avogadro's number (1 mol=6.022×10²³ molecules). Serial dilutions of the samples can be run in order to establish an estimate of the copy numbers. Copy numbers can be normalized to GAPDH or other housekeeping genes to minimize variability in the results due to differences in the RT efficiency and RNA integrity among test samples.

### F. Pharmaceutical Compositions

The present invention also includes pharmaceutical compositions and formulations which include siRNA compounds as described herein. The pharmaceutical compositions can be administered topically, by inhalation, oral or parenteral as taught in U.S. Pat. No. 6,395,545, incorporated herein by reference. A preferred method of administration is as an emulsion or microemulsion. Another method of administration is through use of liposomal formulations. Another method of administration using a “high pressure” delivery of RNAi into mammalian organs may also be used. See Nature Genetics Vol. 32 p107-108 incorporated herein by reference.

### EXAMPLES

Two siRNA oligos which are potent in silencing IKKα and IKKβ mRNA expression were identified. Based on these two potent siRNA duplex oligos, we then designed two hairpin siRNA oligos linking the sense strand and anti-sense strand oligo together with a loop (see FIG. 1 and FIG. 5). The hairpin siRNA oligo was cloned into a retrovirus vector under the human H1 promoter. The retroviruses were produced in Phoenix cells using standard procedures. HeLa cells were infected with the retrovirus expressing IKKα siRNA, IKKβ siRNA or the control GL2 siRNA. HeLa cells infected with IKKβ siRNA repress IKKβ but not IKKα protein expression (FIG. 2) and inhibit TNF-induced IL-6 and IL-8 expression (FIGS. 4A and 4B). Likewise Hela cells infected with IKKα siRNA repress IKKα but not IKKβ protein expression and inhibit TNF-induced IL-6 and IL-8 expression. The retrovirus-infected HeLa cell lines were transfected with the control GL2, or IKKα to and IKKβ siRNA oligos to study the effects of dual siRNA inhibition.

### Example 1

### Preparation of siRNA Duplexes

Approximately 0.2 micromoles of the synthetic siRNAs were obtained from Dharmacon Research Inc. (Lafayette, Colo.). The siRNAs were desalted and deprotected by the supplier and therefore were not further gel purified. The siRNA oligos were annealed and shipped in 4 tubes. 1 ml sterile RNase-free water was added to each tube to make 20 μM siRNA concentrations. After 1 to 2 hours of incubation on ice the siRNAs were ready for use in transfection.

### Example 2

### Construction of the IKKα and IKKβ Retroviral Vectors

To effect the silencing of IKKα and IKKβ, the pSUPER.retro vector is used in concert with a pair of 64-nt oligonucleotides directed to IKKα and IKKβ. SEQ ID. No. 1 and SEQ. ID. No. 2. These were annealed and ligated into the Bgl II/Hind III sites of the pSUPER.retro vector. A pair of control oligos targeting the luciferase gene (SEQ ID. No. 5 and SEQ. ID. No. 6) named as GL-2, was also cloned into the pSUPER.retro vector as a control. Within the 64-nt oligos, the 19-nt target is included in both sense and antisense orientation, separated by a 9-nt spacer sequence. The resulting transcript is predicted to fold back on itself to form a 19-base pair stem-loop structure. The stem-loop precursor transcript is quickly cleaved in the cell to produce a functional siRNA.

Before transfecting the cells with the construct, the presence of the correct inserts was confirmed by sequencing. For a higher rate of stable cell integration, pSUPER.retro can be used with the Phoenix A cell line to produce retroviral supernatants. Cell are cultured in Iscoves MEM supplemented with 10% FBS, 1% Penicillin-Streptomycin, 1% Glutamine, 1% NEAA, 1× Sodium Pyruvate

Phoenix cells are transfected by calcium-phosphate precipitation to produce ecotropic retroviral supernatants. 48 hours to post-transfection, the tissue culture medium is spun at 1,400 rpm for 5 minutes, and the viral supernatant used for infection of human HeLa or HEK293 cells after addition of 4 μg/ml polybrene. Cells are infected for at least 6 hours and allowed to recover for 24 hours in fresh medium. Cells were grown in the presence of puromycin (3 μg/ml for 48 hours).

### Example 3

### Delivery of siRNA to HeLa Cells (Transient Transfection)

Delivery of siRNA duplexes was performed with OLIGOFECTAMINE™ reagent available from Invitrogen (Carlsbad, Calif.). The samples were prepared in a 6 well format. Transfection efficiencies were found to be about 80%.

For each well of a 6 well plate, one tube containing 10 μl of 20 μM siRNA duplex with 90 μl of Opti-MEM, and a separate tube of 4 μl of OLOGOFECTAMINE™ reagent with 96 μl of Opti-MEM were prepared and incubated for 7-10 minutes at room temperature. The content of the two tubes were combined and incubated for another 20 to 25 minutes at room temperature. Then 800 μl of fresh Opti-MEM was added to obtain a final solution of 1000 μl. Then 1000 μl of siRNA-OLIGOFECTAMINE™ was added to cultured cells (40 to 50% confluent). The cells were seeded the previous day in 6-well plates at a density of 2×10⁵ cells/well using 2 ml of DMEM tissue culture medium supplemented with 10% FBS without antibiotics. The control used for transfection was inverted siRNA. A knockdown effect was generally found after 1-2 days.

### Example 4

### Preparation of RNA and PCR Primers

Total RNA was prepared from the cells 2 days after delivery of siRNA's. Total cellular RNA was isolated from tissue or cell samples using the RNeasy™ kit and Rnase-Free DNase Set Protocol from Qiagen (Valencia, Calif.) according to the manufacturer's directions. PCR primers and TaqMan probes were designed using Primer Express 1.5 Software (Applied Biosystems, Foster, Calif.). The sequence of the PCR primers used was SEQ. ID. No. 7: 5′-GCACAGAGATGGTGAAAATCATTG-3′, and SEQ. ID. No. 8: 5′-CAACTTGCTCAAATGACCAAACAG-3′ for IKKα; and SEQ ID. No. 9: 5′-CCGGAAGTACCTGAACCAGTTT-3′ and SEQ ID. No 10: 5′-AGCGCAGAGGCAATGTCACT-3′ for IKKβ. The probe sequence SEQ. ID No. 11: 5′-TGAGCACACGGTCCTGACTCTGCA-3′ for IKKα and SEQ ID. No. 12: 5′-CCTTCCCGCAGACCACAGCAGTTCT-3′ for IKKβ labeled with a reporter fluorescent dye, FAM (6-carboxyfluorescein), at the 5′ end and a fluorescent dye quencher, TAMRA (6-carboxy-tetramethyl-rhodamine), at the 3′ end. The specificity of PCR primers was tested under normal PCR conditions in a thermal cycler prior to TaqMan™ PCR quantitation. Total cellular RNA was isolated from cell samples using the RNeasy Kits and RNase-Free DNase Set Protocol according to the manufacturer's instructions (Qiagen). Reverse transcription (RT) reactions were carried out for each RNA sample in MicroAmp reaction tubes using TaqMan™ reverse transcription reagents. Each reaction tube contained 500 ng of total RNA in a volume of 50 μl containing 1× TaqMan™ RT buffer, 5.5 mM MgCl₂, 500 μM of each dNTP, 2.5 μM of Random Hexamers or oligo-d(T)₁₆ primers, 0.4 U/μl of RNase inhibitor, and 1.25 U/μl of MultiScribe Reverse Transcriptase. RT reactions were carried out at 25° C. for 10 min, 48° C. for 40 min and 95° C. for 5 min Real-time PCR was performed in a MicroAmp Optical 96-Well Reaction Plate (Applied Biosystems). Each well contained 2 μl of each RT product (20 ng total RNA), 1× TaqMan buffer A, 5.5 mM MgCl₂, 200 μM dATP/dCTP/dGTP, 400 μM dUTP, 200 nM primers (forward and reverse), 100 nM TaqMan probe, 0.01 U/μl AmpErase, and 0.025 U/μl AmpliTaq™ Gold DNA polymerase in a total volume of 25 μl. Each well was closed with MicroAmp Optical caps (Applied Biosystems), following complete loading of reagents. Amplification conditions were 2 min at 50° C. (for AmpErase UNG incubation to remove any uracil incorporated into the cDNA), 10 min at 95° C. (for AmpliTaq™ Gold activation), and then run for 40 cycles at 95° C. for 15 s, 60° C. for 1 min. All reactions were performed in the ABI Prism 7700 Sequence Detection System for the test samples, standards, and no template controls. They were run in triplicates using the Sequence Detector V 1.6 program. The Rₙ and Cₜ were averaged from the values obtained in each reaction. A “standard curve” was constructed by plotting the Cₜ vs. the known copy numbers of the template in the standard. According to the standard curve, the copy numbers for all unknown samples were obtained automatically. To determine the copy to numbers of the target transcript, a human genomic DNA (Clontech, Palo Alto, Calif.) was used to generate a standard curve. The copy numbers of genomic DNA template were calculated according to the molecular weight of the human diploid genome [3×10⁹ bp=3×10⁹×660 (M.W.)=2×10¹² g], and then 1 μg/μl genomic DNA was converted into 2.4×10⁶ copy numbers based upon the Avogadro's number (1 mol=6.022×10²³ molecules). The genomic DNA was serially (every ten-fold) diluted at a range of 5×10⁵ to 5×10⁰ copy numbers. Each sample was run in triplicates, and the Rₙ (the ratio of the amount of reporter dye emission to the quenching dye emission) and threshold cycle (Cₜ) values were averaged from each reaction. The copy numbers were then normalized to GAPDH to minimize variability in the results due to differences in the RT efficiency and RNA integrity among test samples.

### Example 5

### Inhibition of NF-κB Dependent Genes by Administration of IKKα and IKKβ Specific siRNA

Table 1 shows the synergistic effect of IKKα and IKKβ specific inhibitors using HeLa cells which stably express IKKα hairpin siRNA (IKKαRV) by retrovirus. The data show that the combination of IKKα and IKKβ siRNA treatment inhibits IL-6 and IL-8 expression more significantly than using either IKKα siRNA or IKKβ siRNA alone.

TABLE 1

| | Level of IL-6 | Level of IL-8 |
|---|---|---|
| | expression | expression |
| Inhibitor | (% of control) | (% of control) |
| IKKα RV + control siRNA | 60% | 66% |
| GL-2 RV + IKKβ siRNA | 45% | 40% |
| IKKα RV + IKKβ siRNA | 30% | 23% |

Table 2 shows the synergistic effect of IKKα and IKKβ specific inhibitors using HeLa cells expressing hairpin IKKαRV siRNA or hairpin IKKβRV siRNA with or without additional IKKα and IKKβ siRNA oligos. The data confirm the result in Table I; that at the background of stable knock-down of either IKKα or IKKβ, combination of IKKα and IKKβ siRNA treatment inhibits IL-6 and IL-8 expression more significantly than using either IKKα siRNA or IKKβ siRNA alone.

TABLE 2

| | Level of IL-6 | Level of IL-8 |
|---|---|---|
| | expression | expression |
| Inhibitor | (% of control) | (% of control) |
| IKKa RV + control siRNA | 45% | 80% |
| IKKb RV + control siRNA | 51% | 21% |
| IKKa RV + IKKb siRNA | 20% | 47% |
| IKKb RV + IKKa siRNA | 36% | 16% |

1. A purified siRNA sequence comprised of SEQ ID. No. 1.

2. An expression vector that expresses the siRNA of claim 1.

3. A retroviral vector that expresses the siRNA of claim 1.

4. The retroviral vector of claim 2 wherein the vector is comprised of a 5′ LTR, a selective gene, a polymerase III, RNA promoter and a 3′LTR.

5. (canceled)

6. A retroviral vector that expresses the siRNA of claim 3.

7. The retroviral vector of claim 6 wherein the vector is comprised of a 5′LTR, a selective agent, promoter and a 3′LTR.

8-25. (canceled)


SEQUENCE LISTING (24 sequences)

SEQ ID NO: 1 - DNA, 64 bp, Artificial Sequence
  Description of Artificial Sequence Synthetic oligonucleotide
gatccccgcagtgcactatgtgtctgttcaagagacagacacatagtgcactgctttttggaaa

SEQ ID NO: 2 - DNA, 64 bp, Artificial Sequence
  Description of Artificial Sequence Synthetic oligonucleotide
agcttttccaaaaagcagtgcactatgtgtctgtctcttgaacagacacatagtgcactgcggg

SEQ ID NO: 3 - DNA, 64 bp, Artificial Sequence
  Description of Artificial Sequence Synthetic oligonucleotide
gatccccggattcagcttctcctaaattcaagagatttaggagaagctgaatcctttttggaaa

SEQ ID NO: 4 - DNA, 64 bp, Artificial Sequence
  Description of Artificial Sequence Synthetic oligonucleotide
agcttttccaaaaaggattcagcttctcctaaatctcttgaatttaggagaagctgaatccggg

SEQ ID NO: 5 - DNA, 64 bp, Artificial Sequence
  Description of Artificial Sequence Synthetic oligonucleotide
gatcccccgtacgcggaatacttcgattcaagagatcgaagtattccgcgtacgtttttggaaa

SEQ ID NO: 6 - DNA, 64 bp, Artificial Sequence
  Description of Artificial Sequence Synthetic oligonucleotide
agcttttccaaaaacgtacgcggaatacttcgatctcttgaatcgaagtattccgcgtacgggg

SEQ ID NO: 7 - DNA, 24 bp, Artificial Sequence
  Description of Artificial Sequence Synthetic primer
gcacagagatggtgaaaatcattg

SEQ ID NO: 8 - DNA, 24 bp, Artificial Sequence
  Description of Artificial Sequence Synthetic primer
caacttgctcaaatgaccaaacag

SEQ ID NO: 9 - DNA, 22 bp, Artificial Sequence
  Description of Artificial Sequence Synthetic primer
ccggaagtacctgaaccagttt

SEQ ID NO: 10 - DNA, 20 bp, Artificial Sequence
  Description of Artificial Sequence Synthetic primer
agcgcagaggcaatgtcact

SEQ ID NO: 11 - DNA, 24 bp, Artificial Sequence
  Description of Artificial Sequence Synthetic probe
tgagcacacggtcctgactctgca

SEQ ID NO: 12 - DNA, 25 bp, Artificial Sequence
  Description of Artificial Sequence Synthetic probe
ccttcccgcagaccacagcagttct

SEQ ID NO: 13 - RNA, 19 nt, Artificial Sequence
  Description of Artificial Sequence Synthetic oligonucleotide
gucuguguaucacgugacg

SEQ ID NO: 14 - RNA, 21 nt, Artificial Sequence
  Description of Artificial Sequence Synthetic oligonucleotide
acucuaucgccagcgugacuu

SEQ ID NO: 15 - RNA, 19 nt, Artificial Sequence
  Description of Artificial Sequence Synthetic oligonucleotide
gcagugcacuaugugucug

SEQ ID NO: 16 - RNA, 19 nt, Artificial Sequence
  Description of Artificial Sequence Synthetic oligonucleotide
ggauucagcuucuccuaaa

SEQ ID NO: 17 - DNA, 2238 bp, Homo sapiens
atggagcggcccccggggctgcggccgggcgcgggcgggccctgggagatgcgggagcggctgggcaccggcggcttcgggaacgtctgtctgtaccagcatcgggaacttgatctcaaaatagcaattaagtcttgtcgcctagagctaagtaccaaaaacagagaacgatggtgccatgaaatccagattatgaagaagttgaaccatgccaatgttgtaaaggcctgtgatgttcctgaagaattgaatattttgattcatgatgtgcctcttctagcaatggaatactgttctggaggagatctccgaaagctgctcaacaaaccagaaaattgttgtggacttaaagaaagccagatactttctttactaagtgatatagggtctgggattcgatatttgcatgaaaacaaaattatacatcgagatctaaaacctgaaaacatagttcttcaggatgttggtggaaagataatacataaaataattgatctgggatatgccaaagatgttgatcaaggaagtctgtgtacatcttttgtgggaacactgcagtatctggccccagagctctttgagaataagccttacacagccactgttgattattggagctttgggaccatggtatttgaatgtattgctggatataggccttttttgcatcatctgcagccatttacctggcatgagaagattaagaagaaggatccaaagtgtatatttgcatgtgaagagatgtcaggagaagttcggtttagtagccatttacctcaaccaaatagcctttgtagtttaatagtagaacccatggaaaactggctacagttgatgttgaattgggaccctcagcagagaggaggacctgttgaccttactttgaagcagccaagatgttttgtattaatggatcacattttgaatttgaagatagtacacatcctaaatatgacttctgcaaagataatttcttttctgttaccacctgatgaaagtcttcattcactacagtctcgtattgagcgtgaaactggaataaatactggttctcaagaacttctttcagagacaggaatttctctggatcctcggaaaccagcctctcaatgtgttctagatggagttagaggctgtgatagctatatggtttatttgtttgataaaagtaaaactgtatatgaagggccatttgcttccagaagtttatctgattgtgtaaattatattgtacaggacagcaaaatacagcttccaattatacagctgcgtaaagtgtgggctgaagcagtgcactatgtgtctggactaaaagaagactatagcaggctctttcagggacaaagggcagcaatgttaagtcttcttagatataatgctaacttaacaaaaatgaagaacactttgatctcagcatcacaacaactgaaagctaaattggagttttttcacaaaagcattcagcttgacttggagagatacagcgagcagatgacgtatgggatatcttcagaaaaaatgctaaaagcatggaaagaaatggaagaaaaggccatccactatgctgaggttggtgtcattggatacctggaggatcagattatgtctttgcatgctgaaatcatggagctacagaagagcccctatggaagacgtcagggagacttgatggaatctctggaacagcgtgccattgatctatataagcagttaaaacacagaccttcagatcactcctacagtgacagcacagagatggtgaaaatcattgtgcacactgtgcagagtcaggaccgtgtgctcaaggagctgtttggtcatttgagcaagttgttgggctgtaagcagaagattattgatctactccctaaggtggaagtggccctcagtaatatcaaagaagctgacaatactgtcatgttcatgcagggaaaaaggcagaaagaaatatggcatctccttaaaattgcctgtacacagagttctgcccggtcccttgtaggatccagtctagaaggtgcagtaacccctcagacatcagcatggctgcccccgacttcagcagaacatgatcattctctgtcatgtgtggtaactcctcaagatggggagacttcagcacaaatgatagaagaaaatttgaactgccttggccatttaagcactattattcatgaggcaaatgaggaacagggcaatagtatgatgaatcttgattggagttggttaacagaatga

SEQ ID NO: 18 - DNA, 3916 bp, Homo sapiens
gagcaggaagtgtttgaggaagtcgcgccgcgctgcccgcgttaagattcccgcattttaatgttttcaggggggtgtcatagccccgggtttggccgccccagccccgccttccccgccccggggagcccgccccctgccccgcgtccctgccgacagagttagcacgacatcagtatgagctggtcaccttccctgacaacgcagacatgtggggcctgggaaatgaaagagcgccttgggacagggggatttggaaatgtcatccgatggcacaatcaggaaacaggtgagcagattgccatcaagcagtgccggcaggagctcagcccccggaaccgagagcggtggtgcctggagatccagatcatgagaaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtggcattcaaaagtgcggcagaagagtgaggtggacattgttgttagcgaagacttgaatggaacggtgaagttttcaagctctttaccctaccccaataatcttaacagtgtcctggctgagcgactggagaagtggctgcaactgatgctgatgtggcacccccgacagaggggcacggatcccacgtatgggcccaatggctgcttcaaggccctggatgacatcttaaacttaaagctggttcatatcttgaacatggtcacgggcaccatccacacctaccctgtgacagaggatgagagtctgcagagcttgaaggccagaatccaacaggacacgggcatcccagaggaggaccaggagctgctgcaggaagcgggcctggcgttgatccccgataagcctgccactcagtgtatttcagacggcaagttaaatgagggccacacattggacatggatcttgtttttctctttgacaacagtaaaatcacctatgagactcagatctccccacggccccaacctgaaagtgtcagctgtatccttcaagagcccaagaggaatctcgccttcttccagctgaggaaggtgtggggccaggtctggcacagcatccagaccctgaaggaagattgcaaccggctgcagcagggacagcgagccgccatgatgaatctcctccgaaacaacagctgcctctccaaaatgaagaattccatggcttccatgtctcagcagctcaaggccaagttggatttcttcaaaaccagcatccagattgacctggagaagtacagcgagcaaaccgagtttgggatcacatcagataaactgctgctggcctggagggaaatggagcaggctgtggagctctgtgggcgggagaacgaagtgaaactcctggtagaacggatgatggctctgcagaccgacattgtggacttacagaggagccccatgggccggaagcaggggggaacgctggacgacctagaggagcaagcaagggagctgtacaggagactaagggaaaaacctcgagaccagcgaactgagggtgacagtcaggaaatggtacggctgctgcttcaggcaattcagagcttcgagaagaaagtgcgagtgatctatacgcagctcagtaaaactgtggtttgcaagcagaaggcgctggaactgttgcccaaggtggaagaggtggtgagcttaatgaatgaggatgagaagactgttgtccggctgcaggagaagcggcagaaggagctctggaatctcctgaagattgcttgtagcaaggtccgtggtcctgtcagtggaagcccggatagcatgaatgcctctcgacttagccagcctgggcagctgatgtctcagccctccacggcctccaacagcttacctgagccagccaagaagagtgaagaactggtggctgaagcacataacctctgcaccctgctagaaaatgccatacaggacactgtgagggaacaagaccagagtttcacggccctagactggagctggttacagacggaagaagaagagcacagctgcctggagcaggcctcatgatgtggggggactcgaccccctgacatggggcagcccatagcaggccttgtgcagtggggggactcgaccccctgacatggggctgcctggagcaggccgcgtgacgtggggctgcctggccgcggctctcacatggtggttcctgctgcactgatggcccaggggtctctggtatccagatggagctctcgcttcctcagcagctgtgactttcacccaggacccaggacgcagccctccgtgggcactgccggcgccttgtctgcacactggaggtcctccattacagaggcccagcgcacatcgctggccccacaaacgttcaggggtacagccatggcagctccttcctctgccgtgagaaaagtgcttggagtacggtttgccacacacgtgactggacagtgtccaattcaaatctttcagggcagagtccgagcagcgcttggtgacagcctgtcctctcctgctctccaaaggccctgctccctgtcctctctcactttacagcttgtgtttcttctggattcagcttctcctaaacagacagtttaattatagttgcggcctggccccatcctcacttcctctttttatttcactgctgctaaaattgtgtttttacctactactttggtggttgtcctcttttcggcaaagttggagcgagtgccaagctctccatctgtggtcctttctgccaagagcgactcatagtaaccaggatgggagagcagctgccttattctgaatcccaaaaattacttgggggtgattgtcacagaggagggacagaaagggtatctgctgaccaccagcctgcctacccatgcccatgtctccattcctgctcaagcgtgtgtgctgggccggggagtccctgtctctcacagcatctagcagtattattaaatggattcattttaaaaatagctcctatattttgtaacatgtctcaaacactcatactgggttccacaatccactgttagaatacctatggttagggcttctgaactaaaataatggaaaattttaacaatttgtatagtgcctggatcattactagtgccataaccctgcttcttcaacatttcacagaacttctcttttatataaaggcaagagcacaaaatgagttcagatgatcacaaacaggtgagttttgttggagaagaaagttggagtaggagactttcacaagtggtttccatggagatagaatgaagcattctgtggtcaagtaagtttagggagctattcatgtttcacttgctttgtggagattcacactatgcactgggaaagtatctgaaaagtcttataataaagaaacaggcttaactttgtgtaagaacactgtttatcaatgtcatttggctatagaaacattttctcctgctgattgtgtgtgtgaaacatgtattaacattccaatgaactagcatttaataaagcacaattttgg

SEQ ID NO: 19 - RNA, 52 nt, Artificial Sequence
  Description of Artificial Sequence Synthetic oligonucleotide
gcagugcacuaugugucuguucaagagacagacacauagugcacugcuuuuu

SEQ ID NO: 20 - RNA, 52 nt, Artificial Sequence
  Description of Artificial Sequence Synthetic oligonucleotide
ggauucagcuucuccuaaauucaagagauuuaggagaagcugaauccuuuuu

SEQ ID NO: 21 - DNA, 64 bp, Artificial Sequence; identical to SEQ ID NO: 1
  Description of Artificial Sequence Synthetic oligonucleotide

SEQ ID NO: 22 - DNA, 63 bp, Artificial Sequence
  Description of Artificial Sequence Synthetic oligonucleotide
gatccccggattcagcttctcctaaattcaagagtttaggagaagctgaatcctttttggaaa

SEQ ID NO: 23 - DNA, 23 bp, Artificial Sequence
  Description of Artificial Sequence Synthetic oligonucleotide
aannnnnnnnnnnnnnnnnnntt

SEQ ID NO: 24 - DNA, 16 bp, Artificial Sequence
  Description of Artificial Sequence Synthetic primer
tttttttttttttttt